(19) United States
(12) Patent Application Publication
(10) Pub. No.: US 2017/0260499 A1
(43) Pub. Date: Sep. 14, 2017

(54) DEVICE FOR THE LONGTERM, UNINTERRUPTED CULTURE, AND OBSERVATION OF EMBRYOS

(71) Applicants: Michael Cecchi, Longboat Key, FL (US); Timothy Schimmel, Randolph, NY (US)

(72) Inventors: Michael Cecchi, Longboat Key, FL (US); Timothy Schimmel, Randolph, NY (US)

(21) Appl. No.: 15/068,556

(22) Filed: Mar. 12, 2016

(51) Int. Cl. C12N 5/073; C12M 1/00; C12M 1/34; C12M 1/32

(52) U.S. Cl. CPC C12N 5/0604; C12M 23/12; C12M 23/34; C12M 41/46

(57) ABSTRACT

A embryo culture dish comprising: a first sidewall; a second sidewall attached to the first sidewall; a third sidewall attached to the second sidewall; a fourth sidewall attached to the third and first sidewalls, the four sidewalls forming an outer perimeter that forms a generally rectangular shape; a dish floor that is generally in communication with the four sidewalls, the dish floor comprising: a top surface that is at a first elevation; at least one rectangular shaped embryo culture well, the at least one rectangular shaped embryo culture well comprising: an outer perimeter that has generally a rectangular shape; a first slanted wall that comprises a first side of the rectangular shaped embryo culture well; a second slanted wall that comprises an opposite side from the first side of the rectangular shaped embryo culture well, the first and second slanted walls extend downward and towards each other until they both intersect with a well floor of the rectangular shaped embryo culture well, the well floor having a well top surface, and where the well top surface is at a second elevation, the second elevation being below the first elevation; at least one shaped well, the shaped well comprising: an outer perimeter that has generally a shape; a shaped well floor, the shaped well floor having a shaped well floor top surface, and where the shaped well floor top surface is at a third elevation, the third elevation being below the first elevation. A method of using a square time-lapse dish, the method comprising: adding a medium to a square time lapse dish; tilting the square time-lapse dish to an angle α; removing excess medium from a large well in the time-lapse dish, with the square time-lapse dish at angle α; and returning the square time-lapse dish to a horizontal orientation.

### TECHNICAL FIELD

This invention relates to a device for the long-term, uninterrupted, culturing of embryos and biological specimens in a controlled incubated environment.

### BACKGROUND

Currently, there are several devices, dishes or holding used in long-term culturing of embryos. Each of these devices is difficult to use and have their own set of problems.

One of these known dishes or holding device is generally used in a device named the Embryoscope®, sold by Vitrolife, AB, (formerly Unisense Fertilitech A/S). The holding device is referred to as an Embryoslide™, which is inserted into the Embryoscope®. The problem that the Embryoslide™ incurs is that when culture medium is placed within the well of the embryoslide, it creates air bubbles. These air bubbles may keep the embryos from resting at the bottom of the well, distort the imaging of the wells by cameras or microscopes, may totally block any imaging of the well and in most cases require intervention by the embryologist. This problem is addressed by the instruction manual which relates to the embryo scope and embryos slide, and is a consistent and repetitive problem in the simple use of this device. This problem may also result in a death of the embryo if in fact the embryo gets caught within the air bubble(s).

Many of the other known time-lapse dishes or devices have small wells, and therefore lead to difficulties with (1) filling the wells with a medium, (2) placing the embryos in the wells, and (3) handling of the embryos with respect to the incubating device.

Other problems with known long term and time-lapse systems are inconsistent temperature in the wells and in the medium, where the embryos sit; and inconsistent temperatures which will create an unfavorable variance in the overall environment the embryo is held in. Known dishes or holding devices may have direct contact with the heating sources which often leads to heat spikes, for instance if the heat source sends a sudden heat surge larger than necessary and leads to overheating.

An additional problem with known dishes is that they often have completely flat bottom surfaces, that is they do not have wells. The flat bottom surface allows the embryo to rest anywhere in the flat bottom, or against a dish wall. This makes locating the embryo with a camera or microscope difficult and time consuming. In addition, if the embryo is against a dish wall, the shape of the embryo may be distorted, thus resulting in inferior imaging.

An additional problem with current dishes and devices for imaging is that the embryo may end up resting in several different locations in the dish and therefore will reduce the ability of the imaging device to use higher magnifications due to location of the embryo. An additional problem with current dishes and imaging is that the embryo may end up resting in locations in the micro drop or well that is unsuitable for proper imaging with a camera or microscope.

Thus there is a need for an invention that overcomes the above listed and other disadvantages.

### SUMMARY OF THE INVENTION

The invention relates to a specimen dish comprising: a first sidewall; a second sidewall attached to the first sidewall; a third sidewall attached to the second sidewall; a fourth sidewall attached to the third and first sidewalls, the four sidewalls forming an outer perimeter that forms a generally rectangular shape; a dish floor that is generally in communication with the four sidewalls, the dish floor comprising: a top surface that is at a first elevation; at least one rectangular shaped embryo culture well, the at least one rectangular shaped embryo culture well comprising: an outer perimeter that has generally a rectangular shape; a first slanted wall that comprises a first side of the rectangular shaped embryo culture well; a second slanted wall that comprises an opposite side from the first side of the rectangular shaped embryo culture well, the first and second slanted walls extend downward and towards each other until they both intersect with a well floor of the rectangular shaped embryo culture well, the well floor having a well top surface, and where the well top surface is at a second elevation, the second elevation being below the first elevation; at least one shaped well, the shaped well comprising: an outer perimeter that has generally a shape; a shaped well floor, the shaped well floor having a shaped well floor top surface, and where the shaped well floor top surface is at a third elevation, the third elevation being below the first elevation.

The invention also relates to a method of using a dish, the method comprising: adding a medium to a dish; tilting the square time-lapse dish to an angle α; removing excess medium from a large well in the dish, with the dish at angle α; and returning the dish to a horizontal orientation.

### BRIEF DESCRIPTION OF THE DRAWINGS

The present disclosure will be better understood by those skilled in the pertinent art by referencing the accompanying drawings, where like elements are numbered alike in the several figures, in which:

FIG. 1 is a top plan view of the disclosed culture dish;

FIG. 2 is a schematic side view of the dish of FIG. 1;

FIG. 3 is a cross-sectional view of the dish of FIG. 1;

FIG. 4 is a another cross-sectional view of the dish of FIG. 1

FIG. 5 is a close up cross-sectional view of a rounded well;

FIG. 6 is a close-up cross-sectional view of a rectangular well;

FIG. 7 shows the disclosed dish filled with media;

FIG. 8 shows the disclosed dish having excess media removed;

FIG. 9 is a flow chart disclosing a method of the invention.

### DETAILED DESCRIPTION

The invention relates to a dish or holding device for the long term culturing of embryos. This invention relates to an improved device and method for growing specimens such as embryos, for an extended period of time, allow excellent visibility for observation with a microscope or camera, allowing time-lapse photography, better and increased magnification of the embryos, to allow their monitoring and to increase the likelihood of more pregnancies and ultimately more live births.

In one embodiment the device may hold up to about 12 embryos and has about 6 additional wells (depressions in the device) for various uses.

The disclosed device may be a dish, or a dish-like configuration that will allow for better imaging of the embryos as they are cultured through a growth period. The disclosed device may provide a suitable location for the long term uninterrupted culturing of a biological specimen such as an embryo. The disclosed device may better protect the embryos safety from deleterious outside influences, such as airborne contaminants, disruptive movement, earthquakes and the like. The disclosed device may provide a method and apparatus of the character described which combine multiple growth and safety features within a single embodiment.

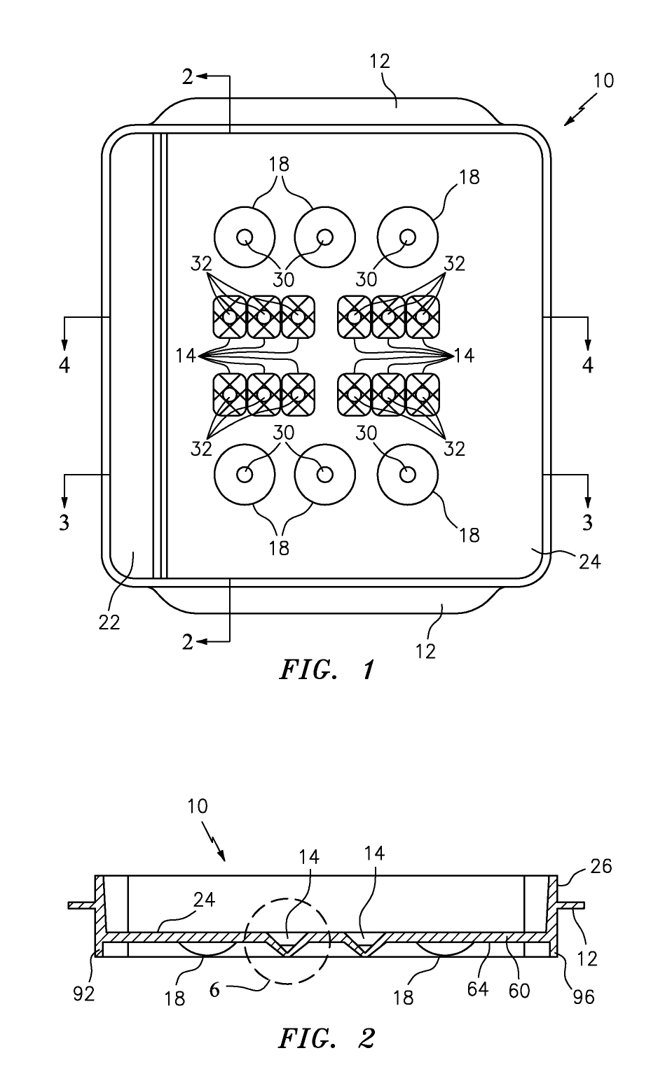

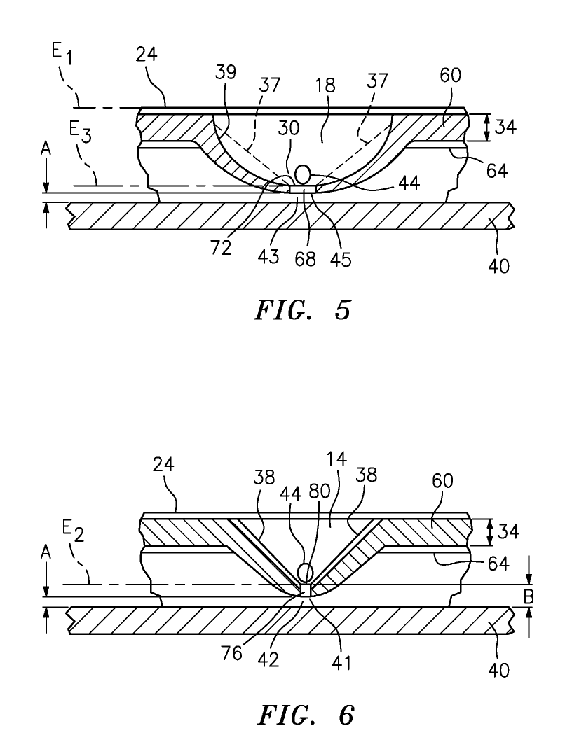

FIG. 1. This is a top view of the invention. The dish 10 may have a generally square or generally rectangular shape with exterior handles 12, which can be used for picking up the dish. The dish comprises a dish floor 60. The dish floor may have a top surface 24, and a bottom surface 64. The top surface 24 is at a first elevation E₁ as shown in FIG. 5. The dish 10 may have about twelve (12) rectangular embryo culture wells 14 which may be in a square or rectangular shape. These wells 14 will contain slanted walls and a flat bottom. The flat bottom results in a flat optically clean area of about 400 micrometers diameter at the base, for the ability to locate and take images of the embryos (not shown) from underneath the dish 10. The dish 10 may also contains six outer wells 18, which may be used for holding other materials, such as oils, media, additional embryos, sperm and the like, and may be used in taking images. Wells 18 may be circular which allows the embryo to sink to the focal point at bottom. The dish 10 may also contain a large well area 22, which may be used to hold additional materials, media solutions. This large well 22, which is recessed from the surface 24 of the dish, and may be used to evacuate the excess media and liquid from the overall dish, allowing the top surface 24 to be generally free of liquids. The dish 10, after the excess liquid is extracted, may then be flooded with mineral oil. The oil is added to cover media solution, to maintain temperature control and to reduce the evaporation of the media solution in the wells. One of ordinary skill in the art will recognize there may be other suitable liquids used in place of mineral oil.

The generally rectangular shape of the wells 14, allows the invention to hold larger quantities of embryo culture media for extended culturing, through to about the blastocyst stage, not requiring the repeated changing out of the media product. The rectangle shape of the wells 14, allows the user to enter the well, with a pipette, from different angles, within the rectangular shape, which may make the wells 14 easier to access and easier to move embryos about.

This dish 10 may have a raised surface 24 or plateau, which is elevated above the wells 14. This raised surface 24, allows the dish to be flooded with media, slightly slanted towards the large well area 22, allowing the wells 14 to retain the media, up to this surface 24, and to be fully filled with media. The excess media may then be drawn out from that large well 22, as necessary. The dish 10 maybe then flooded with mineral oil, above the wells 14 and 18, to protect the media in the wells from evaporation, VOCs and to help maintain the overall temperature of the media and specimens within the wells. The wells 14 and 18 have flat bottoms 32 and 30 respectively. The flat bottoms 30 and 32 may be about 400 micrometers in diameter. This flat surface will allow the embryos to settle to this bottom and allow the embryos to be imaged. This flat surface is engineered to be optically clear and will allow imaging, from below, without distortions.

FIG. 2 is a cross-section view of the invention 10, which shows the protruding handle 12, the side view of the wells 14, and wells 18. Shown in this side view is the raised surface 24, side walls 26, and the thickness of these walls. It illustrates the raised position of the surface 24, in relation to the wells 14 and 18. A well 14 is ‘circled’ and will be further discussed in FIG. 6. A first leg 92 and a second leg 96 are shown extending below the dish floor 60.

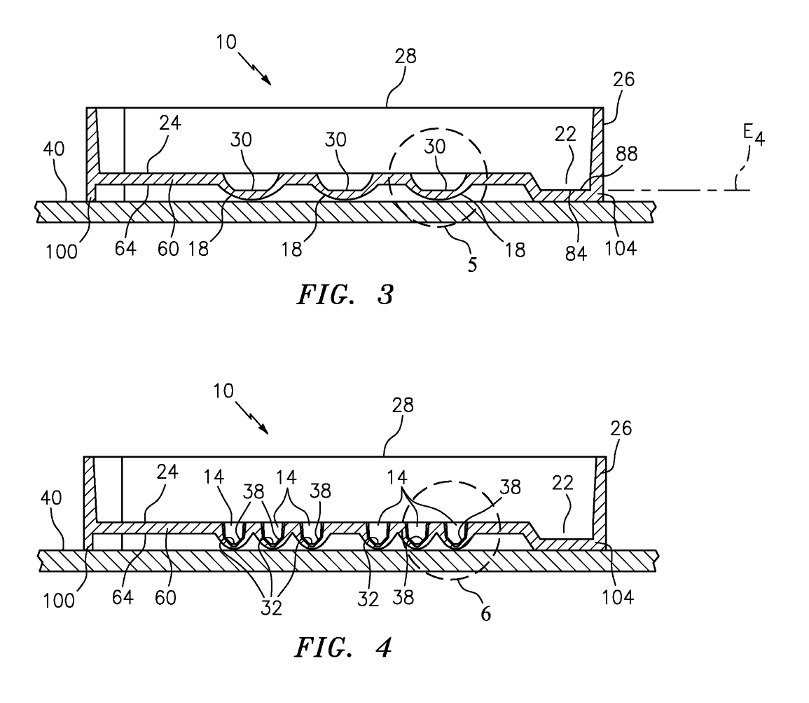

FIG. 3 is a cross-section view of the invention 10 showing the rounded wells 18, of the device 10. Shown are the sidewalls 26 and their thickness, the raised surface 24 and its thickness. Shown is the large well 22, which may be used to remove excess media and liquids. The device 10 may be sitting on a heated surface 40, which may be of glass or metal, with portals to allow the imaging from beneath. Well 18, will be further discussed in FIG. 5. The large well 22 has a well floor 84. The top surface 88 of the well floor 84 is at a fourth elevation E₄. E₄ may be lower than E₁. A second leg 100 and a third leg 104 are shown extending below the dish floor 60.

FIG. 4 shows a cross-sectional view of the invention 10, and more clearly shows the rectangular wells 14, of the device 10. Shown are the sidewalls 26 and their thickness, the raised surface 24 and its thickness. Shown is the large well 22, which may be used to remove excess media and liquids. The device 10 may be sitting on a heated surface 40, which may be of glass or metals, with portals to allow the imaging from beneath. Well 14, will be further discussed in FIG. 6.

FIG. 5 shows a circular well 18 (i.e. the volume which holds the biological specimen) that is above the heating surface 40, and does not come in direct contact with this heated surface. The space 43 between the heated surface 40 and the bottom of the well floor 68 well 18 may be about 2 micrometer. The heated surface 40 may be either glass or metal depending on the usage of the dish. Well 18 may have a semi-spherical shape as shown. Visible in FIG. 5 are optional slanted walls 37 that may be used instead of the curved walls 39. Either type of wall 37, 39 allows the embryo to settle at the lowest point of the well 18. In this illustration the lowest point 45 is flat on both the top and bottom creating an optically clear surface of about 400 μM, which will allow the embryo 44, to be clearly viewed and imaged from beneath. This space 43 is extremely important for the controlling the temperature of the well bottom surface and not to cause overheating and allows a long-term balance. In this view the top surface 24 and the thickness of that surface 34 is shown. The top surface 72 of the well floor 68 is at a third elevation E₃. E₃ may be lower than E₁.

FIG. 6 is a cross-sectional close up view of a well 14. In this view is shown that the well 14 is suspended above the heating surface 40, and does not come in contact with this heated surface. The space 42 between the heated surface 40 and the bottom of the well floor 76 of the well 14 is about 2 micrometer, illustrated by A. This heated surface may be either glass or metal deep pending on the usage of the dish. Visible in this view is the slanted wall 38, which allows the embryo to settle at the lowest point 41 of the well 14. In this illustration the lowest point 41, is flat on both the top and bottom creating an optically clear surface of about 400 μL, which will allow the embryo 44, to be clearly viewed and imaged from beneath. In this view is the top surface 24 and the thickness of that surface 34 is shown. The top surface 80 of the well floor 76 is at a second elevation E₂. E₂ may be lower than E₁.

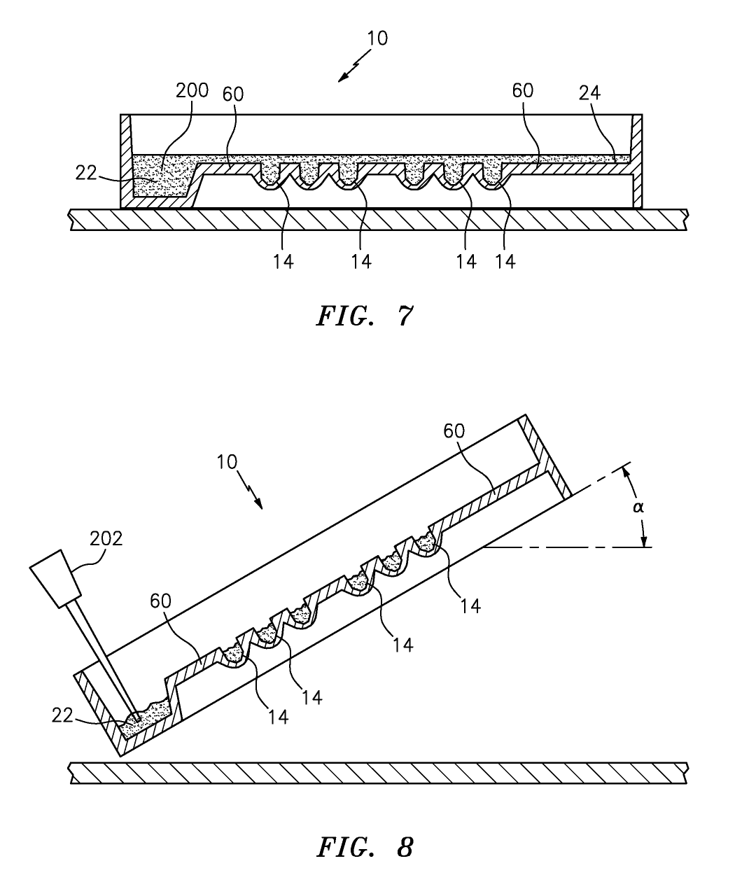

FIG. 7 shows a cross-sectional view of the dish 10 and the wells 14. In this view a culture medium 200 has been added to the dish and has “flooded” the dish. Each well 14 is filled with the medium 200 and with an excess of medium above the wells 14.

FIG. 8, shows dish 10 tilted so that the excess medium can be drawn off with pipette 202 (or other similar device) at well 22. The dish may be slanted from about 1 (one) degree of elevation to about 89 (eighty nine) degrees of elevation to remove the excess media or oils. The dish 10 is shown at an angle α. α is at about 30° in FIG. 8, but can range from about 1° to about 89°. This is a unique feature to this dish and dishes use for embryo culture. This allows a more rapid filing of the wells compared to current methods. After the excess medium 200 is removed, the dish can be un-tilted. In some methods, mineral oil may be added, and the excess mineral oil may be removed by tilting the dish and removing the excess with a pipette 202.

FIG. 9 is a flowchart illustrating a method of using the invention. At act 300 a medium is added to the dish 10. At act 304, the dish 10 is titled to an angle α. At act 308, the excess medium is removed from the large well with the dish at angle α. At act 312, the dish is returned to a horizontal orientation and is ready for use.

The disclosed invention has many advantages. It would be highly desirable to provide a device, which would provide an embryo with the full desired amounts of media to support the growth and to maintain an adequate supply of oil above the embryo to protect it from environment conditions such as VOC's within the incubator, evaporation and contamination. The disclosed dish protects the embryo, allows the embryo to be highly visible by microscope or camera, and allows a variable range of magnifications. The device allows for the long-term culturing of embryos; provide improved images of their development and to be able to implant these embryos. The device may allow a higher resolution in imaging and a more concise series of these images, which will allow better evaluation, better embryo selection and a better likelihood of implanting a more capable embryo for growth, pregnancy and a higher birth rate. The disclosed dish is suitable for taking time-lapse images of the biological specimen. The disclosed dish is configured to keep the wells from directly touching a heat source, which prevents and minimizes heat spikes. The disclosed dish can easily have excess media and other liquids removed by tilting the dish and using a pipette to remove the excess media from the large well 22.

The invention results in an enhanced ability to allow better culturing, viewing and imaging of the embryos using a microscope or camera system, and increases the likelihood of rapid location of the embryos within the designated well. The invention, gives the ability of long term, uninterrupted culturing of the embryos, within this controlled environment, throughout all growth stages up to re-implantation. The inventions allows for a compact apparatus, which can be readily filled with culture medium and/or mineral oil, quickly loaded with embryos, and used in long-term imaging.

It should be noted that the terms “first”, “second”, and “third”, and the like may be used herein to modify elements performing similar and/or analogous functions. These modifiers do not imply a spatial, sequential, or hierarchical order to the modified elements unless specifically stated.

While the disclosure has been described with reference to several embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiments disclosed as the best mode contemplated for carrying out this disclosure, but that the disclosure will include all embodiments falling within the scope of the appended claims.

What is claimed is:

1. An embryo culture dish comprising:
a first sidewall;
a second sidewall attached to the first sidewall;
a third sidewall attached to the second sidewall;
a fourth sidewall attached to the third and first sidewalls, the four sidewalls forming an outer perimeter that forms a generally rectangular shape;
a dish floor that is generally in communication with the four sidewalls, the dish floor comprising:
a top surface that is at a first elevation;
at least one rectangular shaped embryo culture well, the at least one rectangular shaped embryo culture well comprising:
an outer perimeter that has generally a rectangular shape;
a first slanted wall that comprises a first side of the rectangular shaped embryo culture well;
a second slanted wall that comprises an opposite side from the first side of the rectangular shaped embryo culture well, the first and second slanted walls extend downward and towards each other until they both intersect with a well floor of the rectangular shaped embryo culture well, the well floor having a well top surface, and wherein the well top surface is at a second elevation, the second elevation being below the first elevation;
at least one shaped well, the shaped well comprising:
an outer perimeter that has generally a shape;
a shaped well floor, the shaped well floor having a shaped well floor top surface, and wherein the shaped well floor top surface is at a third elevation, the third elevation being below the first elevation.

2. The embryo culture dish of claim 1, wherein the at least one rectangular shaped embryo culture well further comprises:
a third slanted wall in communication with the first and second slanted walls, the third slanted wall comprising a third side of the rectangular shaped embryo culture well;
a fourth slanted wall in communication with the first and second slanted walls, the fourth slanted wall comprising an opposite side from the third side of the rectangular shaped embryo culture well, the third and fourth slanted walls extending downward and towards each other until they both intersect with the well floor of the rectangular shaped embryo culture well.

3. The embryo culture dish of claim 1, wherein the shaped well further comprises:
an shaped outer perimeter that has generally a circular shape; and
a generally semi-spherical shape to the shaped well as the well extends down from the shaped outer perimeter.

4. The embryo culture dish of claim 1, wherein the shaped well further comprises:
an shaped outer perimeter that has generally a circular shape; and
a first slanted wall that comprises a first side of the shaped well, and extends generally from a curved side of the shaped well below the shaped outer perimeter;
a second slanted wall that comprises an opposite side from the first side of the shaped well, the second slanted wall extends generally from a curved side of the shaped well below the shaped outer perimeter, the first and second slanted walls extend downward and towards each other until they both intersect with shaped well floor.

5. The embryo culture dish of claim 1, further comprising:
a large well formed generally adjacent to one of the sidewalls, the large well having a large well floor, and where the top surface of the floor is at a fourth elevation that is generally below the first elevation.

6. The embryo culture dish of claim 1, wherein the large well is at least five times as large in volume as the shaped well.

7. The embryo culture dish of claim 1, further comprising:
a first leg extending downward from the dish floor and extending down past the well floor and the shaped well floor;
a second leg extending downward from the dish floor and extending down past the well floor and the shaped well floor; and
wherein the legs are configured to hold the embryo culture dish above a horizontal surface such that the well floor and the shaped well floor both will not touch the horizontal surface.

8. The embryo culture dish of claim 1, further comprising:
a first flat surface located on the top surface of the well floor,
a second flat surface located on the bottom surface of the well floor, the second and first flat surfaces are parallel to each other and horizontal when the embryo culture dish is in an in-use orientation.

9. The embryo culture dish of claim 1, further comprising:
a third flat surface located on the top surface of the shaped well floor,
a fourth flat surface located on the bottom surface of the shaped well floor, the third and fourth flat surfaces are parallel to each other and horizontal when the embryo culture dish is in an in-use orientation.

10. The embryo culture dish of claim 1, further comprising:
at least eleven additional rectangular shaped embryo culture wells.

11. The embryo culture dish of claim 1, further comprising:
at least five additional shaped wells.

12. The embryo culture dish of claim 1, further comprising:
at least one additional rectangular shaped embryo culture well.

13. The embryo culture dish of claim 1, further comprising:
at least one additional shaped well.

14. A method of using an embryo culture dish, the method comprising:
adding a medium to a square time lapse dish;
tilting the square time-lapse dish to an angle α;
removing excess medium from a large well in the time-lapse dish, with the square time-lapse dish at angle α; and
returning the square time-lapse dish to a horizontal orientation.
